(19) United States
(12) Patent Application Publication
(10) Pub. No.: US 2019/0241864 A1
(43) Pub. Date: Aug. 8, 2019

(54) Composition Comprising Non-Germinant and Bacterial Spores

(71) Applicant: NOVOZYMES BIOAG A/S, Bagsvaerd (DK)

(72) Inventor: Jared Heffron, Salem, VA (US)

(73) Assignee: NOVOZYMES BIOAG A/S, Bagsvaerd (DK)

(21) Appl. No.: 16/316,782

(22) PCT Filed: Jun. 23, 2017
(86) PCT No.: PCT/US2017/038927

§ 371 Date: Jan. 10, 2019

Related U.S. Application Data
(60) Provisional application No. 62/369,096, filed on Jul. 30, 2016.

(51) Int. Cl. C12N 1/20; C12N 1/38

(52) U.S. Cl. CPC C12N 1/20; C12N 1/38

(57) ABSTRACT

Composition comprising bacterial spore and its non-germinant (preferably amino acid, more preferably valine or aspargine) which alone in water does not cause germination of said spore, but accelerate its germination in combination with known germinants of said spore.

### BACKGROUND

Germination of bacterial spores to vegetative cells can be caused by germinant molecules when they contact the spores. Even with sufficient germinants, however, germination of bacterial spores may be inefficient in some cases. For example, some spores in a population may not germinate when contacted with germinants. A better understanding of bacterial spore germination may provide for more efficient germination of bacterial spore populations.

### SUMMARY

Germinant molecules, alone or in combination with other germinants, are able to cause germination of bacterial spores. We found that some molecules without demonstrable germinant activity on certain spores can, when contacted with the spores, increase the efficiency of subsequent spore germination caused by germinants. These non-germinant molecules, therefore, can enhance spore germination caused by germinants.

In one example, for a population of bacterial spores from a strain of Bacillus that germinates when contacted with a first amino acid and a sugar, a second amino acid, having no demonstrable germinant activity on these spores, could be added to the spores prior to germination. We showed that, when these spores were subsequently contacted with the first amino acid and sugar to cause germination, a greater percentage of the spores germinated as compared to the same spores that had not been pretreated with the second amino acid. Generally, the non-germinant molecules are in contact with the spores when the germinants are added.

These findings indicate that pretreatment of bacterial spores with certain non-germinant molecules may cause increased germination of bacterial spores, or may even provide for germination of bacterial spores that would otherwise not germinate. For spore-containing bacterial products that, after dispersal into an environment, need to germinate to vegetative cells to provide a desired effect, the compositions and methods disclosed herein may provide increased effectiveness of these products.

Disclosed herein are compositions, methods, and kits related to addition of non-germinant molecules to bacterial spores to affect efficiency of spore germination.

### BRIEF DESCRIPTION OF THE DRAWINGS

In the accompanying drawings, which are incorporated in and constitute a part of the specification, embodiments of compositions, methods, and kits related to addition of non-germinant molecules to bacterial spores are illustrated which, together with the detailed description given below, serve to describe the examples. It will be appreciated that the embodiments illustrated in the drawings are shown for the purpose of illustration and not for limitation. It will be appreciated that changes, modifications, and deviations from the embodiments illustrated in the drawings may be made without departing from the spirit and scope of the invention, as disclosed below.

FIG. 1 A-C illustrates example spore germination data. (A) shows example germination kinetics, as determined from measurement of dipicolinic acid release from spores. T_{lag}, Gₘₐₓ, germination heterogeneity, and Gᵣₐₜₑ are shown on the germination curves. In (B), the dotted line shows more rapid initiation of germination (decreased T_{lag}), an increased rate of germination (increased Gᵣₐₜₑ), decreased germination heterogeneity, and increased number of spores that germinated (increased Gₘₐₓ), as compared to the solid line. In (C), the dotted line shows an increased Gₘₐₓ and increased germination heterogeneity, but no effect on T_{lag} or Gᵣₐₜₑ.

FIG. 2 illustrates example data from a spore germination experiment. Spores from Bacillus amyloliquefaciens strain SB3615 were used. Decrease in relative optical density (OD) indicates germination of spores. Spores were incubated in either brain-heart infusion medium (●), L-alanine (▴), or buffer alone (♦).

FIG. 3 illustrates example data from a spore germination experiment. Spores from Bacillus pumilis strain SB3189 were used. Decrease in relative optical density (OD) indicates germination of spores. Spores were incubated in either brain-heart infusion medium (●), L-alanine+D-fructose (▴), L-cysteine+D-fructose (▪), or buffer alone (♦). The symbols representing L-alanine+D-fructose (▴) obscure the symbols for L-alanine+sucrose (traces are nearly the same). Likewise, the symbols for L-cysteine+D-fructose (▪) obscure the symbols for L-cysteine+sucrose (traces are nearly the same).

FIG. 4 illustrates example data from a spore germination experiment. Spores from Bacillus megaterium strain SB3112 were used. Decrease in relative optical density (OD; ●), and increase in relative fluorescence (due to DPA release from spores; ◯) indicate germination of spores. Spores were incubated in 20 mM L-proline, 20 mM D-glucose, and 50 mM KBr. Each error bar represents a standard deviation obtained from at least three independent measurements.

FIG. 5 illustrates example data from a spore germination experiment. Spores from Bacillus pumilus strain SB3189 were used. Percent germination of spores is indicated on the y-axis. Spores were germinated with 10 mM AAGFK after initial incubation at 22° C. with L-valine at concentrations of 0 (♦), 0.5 (▪), 3.0 (▴), or 10.0 (x) mM.

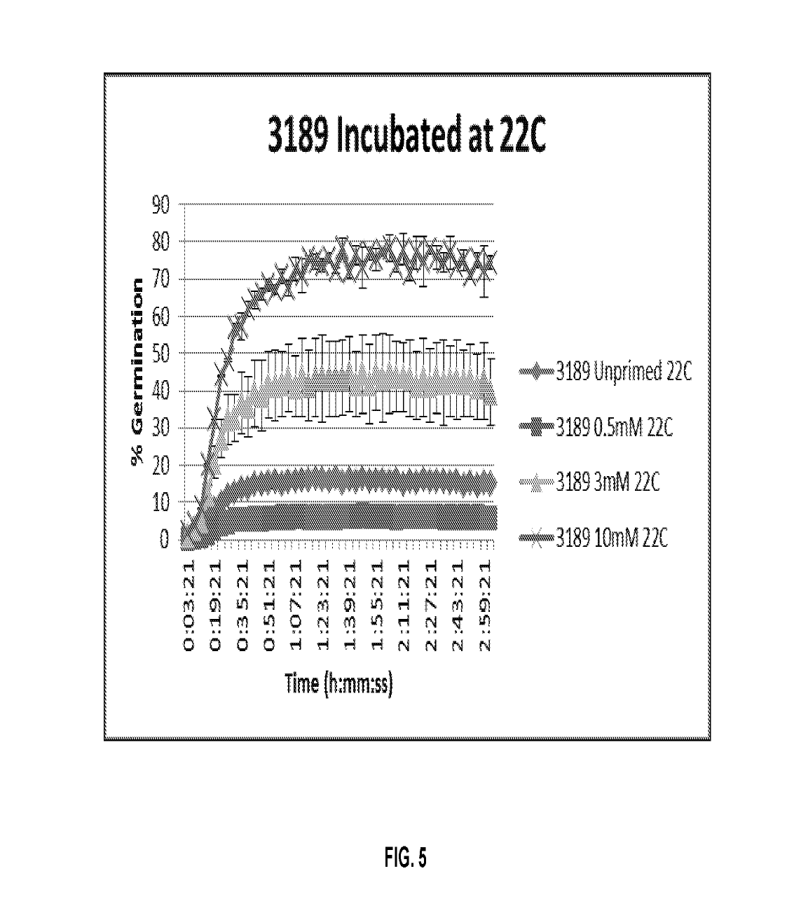

### DETAILED DESCRIPTION

### Definitions

The following includes definitions of selected terms that may be used throughout the disclosure and in the claims. The definitions include various examples and/or forms of components that fall within the scope of a term and that may be used for implementation. The examples are not intended to be limiting. Both singular and plural forms of terms fall within the definitions.

As used herein, “able to germinate,” in reference to bacterial spores, means that at least some of the spores in a population will germinate when provided with sufficient germinants.

As used herein, “about” means±10% with respect to the stated value or property.

As used herein, “add” means to put something together with something else.

As used herein, “additive,” when referring to effects of molecules on parameters of spore germination, means that the effects of a combination of molecules on a germination parameter is generally about the same as the sum of effects of the individual molecules of the combination. The combination of molecules producing this effect may be called an additive combination.

As used herein, “after” means following.

As used herein, “alone” generally refers to whether germinants can cause germination of bacterial spores without the presence of other germinants. Germinants that alone can cause germination can cause germination without other germinants. Germinants that alone cannot cause germination, cannot, at least at a specific concentration, cause germination without other germinants. “Alone” may also refer to non-germinants used without the presence of other non-germinants.

As used herein, “bacteria” means prokaryotic organisms that have peptidoglycan in their cell walls, and have lipids that contain fatty acids in their membranes.

As used herein, “bacterial spores” refers to the structures formed by some bacteria during a process called sporulation. Generally, bacterial spores are resistant to environmental conditions, metabolically inactive, and unable to reproduce. Bacterial spores are generally able to germinate into vegetative cells.

As used herein, “capable of” refers to the ability or capacity to do or achieve a specific thing (e.g., ability of spores to germinate).

As used herein, “cause,” when used as a verb, means to make something happen.

As used herein, “combination” means things that are in proximity to one another or used together. For example, when a first germinant is in combination with a second germinant, the first and second germinants are in proximity to one another or used together.

As used herein, “compared to” means measurement of similarity or dissimilarity between things.

As used herein, “concentration” means an amount of something in a given volume.

As used herein, “contacting” means an act to cause things to physically touch. As used herein, “contact,” with reference to two or more things, means that the things physically touch each other.

As used herein, “contain” means to have or hold something within.

As used herein “contextual” is used to describe certain non-germinant molecules. Generally, a contextual non-germinant has been shown to have no germinant activity on certain spores using the same assay and assay conditions as those used to show that other molecules have germinant activity on the spores.

As used herein “contribute to” means help to cause or bring about something; to facilitate something.

As used herein, “decrease” means to make or become smaller, fewer, or less.

As used herein, “different” means not the same as.

As used herein, “disperse” means to distribute or spread over an area.

As used herein, “dry” means free from liquid or moisture. Generally, a thing may be classified as dry based on moisture content.

As used herein, “efficiency,” may be used to describe germination of one population of spores as compared to a second population of spores. In some examples, a first population of germinating spores may be said to germinate with higher efficiency or more efficiently than a second population of germinating spores if, for example, the first population has a decreased T_{lag}, decreased germination heterogeneity, increased Gₘₐₓ, or increased Gᵣₐₜₑ as compared to the second population.

As used herein, “endospore” means a type of spore that develops inside of bacteria.

As used herein, “environment” means a particular physical location and/or set of conditions.

As used herein, “form,” when used as a verb, means to create something.

As used herein, “from” means the source of something.

As used herein, “germinant” means molecules that, alone or in combination with other germinants, generally at specific concentrations, have the ability to cause bacterial spores to germinate. When a germinant is defined as a germinant because it can cause bacterial spores to germinate in combination with one or more other germinants (i.e., the germinant is unable to cause spore germination by itself), the one or more other germinants in the combination also cannot alone, under the conditions tested, cause germination of the spores. Herein, water is not considered a germinant (i.e., the term “germinant” does not encompass water). A set of one or more germinants, at specific concentrations, that can cause germination of a population of bacterial spores, may be said to be a full complement or complete set of germinants. A set of one or more germinants, at specific concentrations, that do not cause germination of a population of bacterial spores, but that generally do cause germination in combination with one or more other germinants, may be said to be a partial complement or incomplete set of germinants.

As used herein, “germinate” refers to the process whereby a bacterial spore becomes a vegetative cell.

As used herein, “germination heterogeneity” refers to the window of time over which a population of spores germinates after receiving a stimulus sufficient to cause germination. Generally, this period of time begins when the T_{lag} period ends, and ends when the Gₘₐₓ is first reached. Germination heterogeneity is a germination parameter.

As used herein, “germination parameter” refers to a measurable factor describing germination of a population of bacterial spores. Example germination parameters include Gₘₐₓ, T_{lag}, germination heterogeneity, and Gᵣₐₜₑ.

As used herein, “Gₘₐₓ” refers to the percentage of bacterial spores within a population of spores that germinate. Gₘₐₓ is a germination parameter.

As used herein, “Gᵣₐₜₑ” refers to the rate at which spores germinate to vegetative cells and is generally visualized as the slope of the linear part of a germination curve (i.e., plot of germination over time). Gᵣₐₜₑ is a germination parameter

As used herein, “gram-positive” refers to bacteria that stain a certain way in a Gram stain procedure. Generally, gram-positive bacteria differ in their structure and/or arrangement of cellular membrane and cell wall as compared to gram-negative bacteria.

As used herein, “heat activate” refers to treatment of bacterial spores at a specific temperature for a specific period of time. Generally, bacterial spores are heat activated after a population of bacteria has substantially finished sporulating. In some examples, heat activation of spores may affect parameters of subsequent spore germination (e.g., increase efficiency of germination).

As used herein, “increase” means to make or become larger, greater, or more.

As used herein, “initial,” with reference to addition of germinants to bacterial spores, refers to one or more additions of germinants to bacterial spores that do not cause germination. Generally, germination may be caused by a “subsequent” addition of germinants to the spores. “Initial” may also refer to addition of a non-germinant to bacterial spores prior to subsequent addition of germinants to the spores.

As used herein, “kit” refers to a set or collection of two or more things, generally for use in a purpose. The two or more things that are part of a kit may be said to be “packaged” into or as a kit.

As used herein, “knowledge” means facts or information acquired by a person.

As used herein, “known” means recognized or within the scope of knowledge.

As used herein, “liquid,” refers to a state of matter that flows freely, has a definite volume and no fixed shape (e.g., it takes the shape of a container in which it is housed). Example liquids include, without limitation, emulsions, solutions, and suspensions.

As used herein, “mixture” means a combination of different things that are individually distinct. Herein, a mixture may be dry, moist, wet, or liquid.

As used herein, “moisture content” means the amount of water in a sample. Herein, moisture content is determined on a wet basis (i.e., mass of water in a sample/total mass of sample). For example, a sample with mass 10 grams, 1 gram of which is water, has a moisture content of 0.1 or 10%.

As used herein, “molecule” refers to two or more atoms held together by chemical bonds.

As used herein, “non-germinant” means molecules that have no known or demonstrable germinant activity, at least within the context of the testing performed. Herein, water is not considered a non-germinant (i.e., the term “non-germinant” does not encompass water). Non-germinant activity is defined with respect to specific spores. A molecule that lacks demonstrable germinant activity on spores from one strain of bacteria, may have germinant activity on spores from another strain of bacteria. Herein, non-germinants are defined using an assay of bacterial spores in water, where substances to be tested for germinant activity are added to the mixture of the spores in water. Examples of assays like this are described herein in Example 2.

As used herein, “not present” means absent.

As used herein, “population” means a collection of things (e.g., bacterial spores) or totality of things in a group. In some examples a population of bacterial spores may be a stable population.

As used herein, “possess” means to control or hold.

As used herein, “present” means to exist in a particular location.

As used herein, “pretreatment,” with reference to bacterial spores, refers to doing something to the spores prior to germination of the spores. In some examples, spores are pretreated with (i.e., contacted with) one or more non-germinant molecules prior to germination.

As used herein, “prior” means before.

As used herein, “rate-limiting” generally refers to component that controls the outcome of a process. For example, a germinant may be said to be rate-limiting when a parameter of germination (e.g., Gₘₐₓ) is proportional to the concentration of the germinant.

As used herein, “set” means a group or collection of things. In some examples, a set of germinants may contain 1 or more germinants.

As used herein, “simultaneous” means at the same time.

As used herein, “single” means one.

As used herein, “solid,” refers to a state of matter that possesses structural rigidity and resistance to changes in shape or volume. Example solids include, without limitation, crystals, dusts, granules, gels, pastes, pellets, pressings, powders, and tablets.

As used herein, “specific” means particular or clearly identified.

As used herein, “stable,” when referring to a population of bacterial spores, means that the bacterial spores in the population generally are not undergoing germination. A stable population of bacterial spores may be capable of germinating or able to germinate.

As used herein, “subsequent,” with reference to addition of germinants to bacterial spores, refers to an addition of germinants to bacterial spores that occurs after one or more “initial” additions of germinants or non-germinants to the spores. Generally, the subsequent addition of germinants causes germination.

As used herein, “synergy,” when referring to effects of molecules on parameters of spore germination, means that the effects of a combination of molecules on a germination parameter are generally greater than the sum of effects of the individual molecules of the combination. This effect may be called a synergistic effect. A combination of two or more molecules with greater than additive effects may be called a synergistic combination.

As used herein, “substance” means a particular thing with uniform properties.

As used herein, “sufficient” means enough or adequate. Sufficient germinants means germinants that are able to cause germination of specific spores.

As used herein, “T_{lag}” means the duration between the time when a population of bacterial spores receives a stimulus sufficient to cause germination and the time when spores in the population begin to germinate. T_{lag} is a germination parameter.

As used herein, “vegetative cells” refers to bacterial cells that are metabolically active and/or actively growing/dividing. Vegetative bacterial cells are not spores.

As used herein, “with” means accompanied by.

### Bacterial Spores

Some gram-positive bacteria may form bacterial spores or endospores under certain conditions. An example condition under which vegetative cells of bacteria form spores may be limiting amounts of nutrients needed for vegetative growth of the bacteria. Methods for obtaining bacterial spores from vegetative cells are well known in the field. In some examples, vegetative bacterial cells are grown in liquid medium. Beginning in the late logarithmic growth phase or early stationary growth phase, the bacteria may begin to sporulate. When the bacteria have finished sporulating, the spores may be obtained from the medium, by using centrifugation for example. Various methods may be used to kill or remove any remaining vegetative cells. Various methods may be used to purify the spores from cellular debris and/or other materials or substances. Some example methods for producing bacterial spores are described in Example 1 of this disclosure. Bacterial spores may be differentiated from vegetative cells using a variety of techniques, like phase-contrast microscopy or tolerance to heat, for example.

Bacterial spores are generally environmentally-tolerant structures that are metabolically inert or dormant. Sometimes, because of their environmental tolerance, bacterial spores are chosen to be used in commercial microbial products. These products may be designed to be dispersed into an environment where the spores will germinate into vegetative cells and perform an intended function.

A variety of different bacteria may form spores. Bacteria from any of these groups may be used in the compositions, methods, and kits disclosed herein. For example, some bacteria of the following genera may form endospores: Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, and/or Vulcanobacillus.

In some examples, the bacteria that may form endospores are from the genus Bacillus. In various examples, the Bacillus bacteria may be strains of Bacillus alcalophilus, Bacillus alvei, Bacillus aminovorans, Bacillus amyloliquefaciens, Bacillus aneurinolyticus, Bacillus aquaemaris, Bacillus atrophaeus, Bacillus boroniphilius, Bacillus brevis, Bacillus caldolyticus, Bacillus centrosporus, Bacillus cereus, Bacillus circulans, Bacillus coagulans, Bacillus firmus, Bacillus flavothermus, Bacillus fusiformis, Bacillus globigii, Bacillus infernus, Bacillus larvae, Bacillus laterosporus, Bacillus lentus, Bacillus licheniformis, Bacillus megaterium, Bacillus mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophillus, Bacillus subtilis, Bacillus thermoglucosidasius, Bacillus thuringiensis, Bacillus vulgatis, Bacillus weihenstephanensis, or combinations thereof.

In some examples, the bacterial strains that form spores may be strains of Bacillus, including: Bacillus pumilus strain NRRL B-50016; Bacillus amyloliquefaciens strain NRRL B-50017; Bacillus amyloliquefaciens strain PTA-7792 (previously classified as Bacillus atrophaeus); Bacillus amyloliquefaciens strain PTA-7543 (previously classified as Bacillus atrophaeus); Bacillus amyloliquefaciens strain NRRL B-50018; Bacillus amyloliquefaciens strain PTA-7541; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136 (also known as DA-33R, ATCC accession No. 55406); Bacillus amyloliquefaciens strain NRRL B-50141; Bacillus amyloliquefaciens strain NRRL B-50399; Bacillus licheniformis strain NRRL B-50014; Bacillus licheniformis strain NRRL B-50015; Bacillus amyloliquefaciens strain NRRL B-50607; Bacillus subtilis strain NRRL B-50147 (also known as 300R); Bacillus amyloliquefaciens strain NRRL B-50150; Bacillus amyloliquefaciens strain NRRL B-50154; Bacillus megaterium PTA-3142; Bacillus amyloliquefaciens strain ATCC accession No. 55405 (also known as 300); Bacillus amyloliquefaciens strain ATCC accession No. 55407 (also known as PMX); Bacillus pumilus NRRL B-50398 (also known as ATCC 700385, PMX-1, and NRRL B-50255); Bacillus cereus ATCC accession No. 700386; Bacillus thuringiensis ATCC accession No. 700387 (all of the above strains are available from Novozymes, Inc., USA); Bacillus amyloliquefaciens FZB24 (e.g., isolates NRRL B-50304 and NRRL B-50349 TAEGRO® from Novozymes), Bacillus subtilis (e.g., isolate NRRL B-21661 in RHAPSODY®, SERENADE® MAX and SERENADE® ASO from Bayer CropScience), Bacillus pumilus (e.g., isolate NRRL B-50349 from Bayer CropScience), Bacillus amyloliquefaciens TrigoCor (also known as “TrigoCor 1448”; e.g., isolate Embrapa Trigo Accession No. 144/88.4Lev, Cornell Accession No. Pma007BR-97, and ATCC accession No. 202152, from Cornell University, USA) and combinations thereof.

In some examples, the bacterial strains that form spores may be strains of Bacillus amyloliquefaciens. For example, the strains may be Bacillus amyloliquefaciens strain PTA-7543 (previously classified as Bacillus atrophaeus), and/or Bacillus amyloliquefaciens strain NRRL B-50154, Bacillus amyloliquefaciens strain PTA-7543 (previously classified as Bacillus atrophaeus), Bacillus amyloliquefaciens strain NRRL B-50154, or from other Bacillus amyloliquefaciens organisms.

In some examples, the bacterial strains that form spores may be Brevibacillus spp., e.g., Brevibacillus brevis; Brevibacillus formosus; Brevibacillus laterosporus; or Brevibacillus parabrevis, or combinations thereof.

In some examples, the bacterial strains that form spores may be Paenibacillus spp., e.g., Paenibacillus alvei; Paenibacillus amylolyticus; Paenibacillus azotofixans; Paenibacillus cookii; Paenibacillus macerans; Paenibacillus polymyxa; Paenibacillus validus, or combinations thereof.

Bacterial spores used in the compositions, methods, and kits disclosed herein may or may not be heat activated. In some examples, the bacterial spores are heat activated. In some examples, the bacterial spores are not heat inactivated.

For the compositions, methods, and kits disclosed here, populations of bacterial spores are generally used. In some examples, a population of bacterial spores may include bacterial spores from a single strain of bacterium. In some examples, a population of bacterial spores may include bacterial spores from 2, 3, 4, 5, or more strains of bacteria. Generally, a population of bacterial spores contains a majority of spores and a minority of vegetative cells. In some examples, a population of bacterial spores does not contain vegetative cells. In some examples, a population of bacterial spores may contain less than about 1%, 2%, 3%, 4%, 5%, 6%, 7%, 8%, 9%, 10%, 15%, 20%, 25%, 30%, 40%, or 50% vegetative cells, where the percentage of bacterial spores is calculated as ((vegetative cells/(spores in population+vegetative cells in population))×100). Generally, populations of bacterial spores used in the disclosed compositions, methods, and kits, are stable (i.e., not undergoing germination), with at least some individual spores in the population capable of germinating.

Populations of bacterial spores used in this disclosure may contain bacterial spores at different concentrations. In various examples, populations of bacterial spores may contain, without limitation, at least 1×10², 5×10², 1×10³, 5×10³′ 1×10⁴, 5×10⁴′ 1×10⁵, 5×10⁵′ 1×10⁶, 5×10⁶′ 1×10⁷, 5×10⁷′ 1×10⁸, 5×10⁸′ 1×10⁹, 5×10⁹, 1×10¹⁰, 5×10¹°, 1×10¹¹, 5×10¹¹, 1×10¹², 5×10¹², 1×10¹³, 5×10¹³, 1×10¹⁴, or 5×10¹⁴ spores/ml or spores/cm³.

### Germination of Spores

Once formed, bacterial spores can exist as spores indefinitely. However, if bacterial spores receive sufficient stimuli, they may germinate to become vegetative cells. Such stimuli may be said to cause germination. Generally, the stimuli that cause germination of spores include substances, molecules for example, whose presence, and possibly concentration, may be sensed or detected by the spores. Some germinants, referred to as nutrient germinants, are sensed when they interact with receptors in the inner membrane of the spores. Other germinants, referred to as non-nutrient germinants, are sensed by spores independent of receptors. Generally, germinants contact bacterial spores to cause germination.

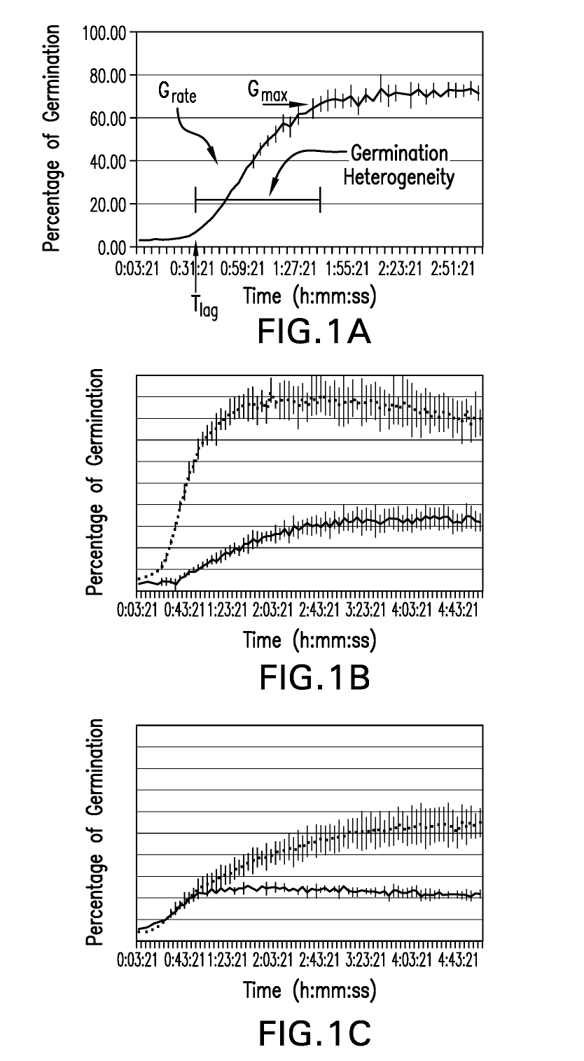

After a population of spores receives a stimulus sufficient to cause germination, germination of the population of spores may be heterogeneous. For example, individual spores within a spore population may germinate at different times after a stimulus sufficient to cause germination is received by the spores. The duration between the time that a sufficient stimulus occurs and the time when spores in a population begin to germinate is called T_{lag} (FIG. 1). The duration between the time when spores in the population begin to germinate and the time when spores cease to germinate is called germination heterogeneity (e.g., the window of time over which germination occurs; FIG. 1). The rate at which spores in the population germinate is called Gᵣₐₜₑ and is generally equivalent to the slope of the linear portion of a germination curve or plot (FIG. 1). Often, not all spores in a population will germinate after a stimulus sufficient to cause germination is received. The percentage of spores in a population that do germinate is called Gₘₐₓ (FIG. 1). All of these measurements—T_{lag}, germination heterogeneity, Gᵣₐₜₑ, and Gₘₐₓ—are parameters or characteristics that describe germination of the population of spores and are called germination parameters. Other germination parameters may exist. A first population of spores that germinates with a decreased T_{lag}, decreased germination heterogeneity, increased Gᵣₐₜₑ, or increased Gₘₐₓ, as compared to a second population of spores, may be said to germinate more efficiently than the second population of spores.

The process of germination may be measured or followed using a variety of methods. For example, bacterial spores appear shiny, bright, or refractile when viewed through a phase-contrast microscope, while vegetative bacterial cells appear dark or non-refractile. Bacterial spores release dipicolinic acid (DPA) when germination is caused. DPA release by spores can be measured. These methods are described and used in some of the studies described in the Examples of this disclosure. Other methods for measuring germination of bacterial spores are known in the field and can be used.

### Germinants

A variety of events can cause bacterial spores to germinate. In some examples, substances that are molecules can cause germination. Generally, these molecules are called germinants. Germinants can, either alone or in combination with other germinants, cause germination of bacterial spores. When molecules are defined as causing spore germination when in a combination with other molecules, the individual molecules of the combination do not cause germination alone. The germinants may have to be present at certain concentrations in order to cause germination. In some examples herein, when a germinant or set of germinants is said to “cause germination,” it means that the one or more germinants, when contacted with a population of spores, results in at least some of the spores in the population becoming vegetative bacterial cells. A single germinant that causes germination, or a combination of germinants that cause germination, may be said to be “sufficient” to cause germination, or may be referred to as a full complement or complete set of germinants. Single germinants or combinations of germinants that do not cause germination, may be referred to as partial complements or incomplete sets of germinants.

The molecules or combinations of molecules that cause germination of specific populations of spores may vary. For example, a single amino acid may cause spores from one species of bacteria to germinate, while an amino acid and a sugar, a sugar and a salt, or a sugar, salt, and an amino acid may be needed to cause germination of spores from another species. The molecules that cause germination of spores may be specific. For example, if an amino acid causes germination, it may be a specific amino acid. In other examples, the specificity may be less pronounced. For example, for some spores, if an amino acid causes germination, a number of amino acids may substitute for one another.

Spores from different strains of the same bacterial species may germinate under different conditions. For example, spores from one strain may need only L-alanine to germinate while spores from a second strain may need L-cysteine plus sucrose. Generally, the specific molecules or combination of molecules that cause germination are specific to a strain. Generally, the molecules that cause germination can be empirically determined.

Related to specificity and substitution of one germinant for another is the finding that spores from a single bacterium may have more than one germinant or combination of germinants that can cause germination. For example, spores from the same bacterium may germinate after contact with L-alanine plus D-fructose, L-histidine plus D-fructose, or L-leucine plus D-fructose. Some examples of this can be seen in Table 2 in this disclosure.

Germinants may have to contact bacterial spores at certain concentrations to cause germination. In some examples, a germinant may not cause germination when present below a certain concentration, but may cause germination above that concentration. In some examples, a germinant may cause germination when present below a certain concentration, but may not cause germination above that concentration. In some examples, too low of a germinant concentration, or too high of a germinant concentration may not cause germination—the germinant concentration may have to be within a range to cause germination.

In some examples, germinants may be present in compositions disclosed herein at concentrations of between about 0.001 mM-10.0 M, 0.01 mM-5.0 M, 0.1 mM-1.0 M, or 1.0 mM-0.1 M. In some examples, the germinants may be present in compositions disclosed herein at concentrations of about 0.01, 0.05, 1, 2, 3, 4, 5, 6, 7, 8, 10, 15, 20, 25, 30, 35, 40, 45, 50, 60, 70, 80, 90, or 100 mM. Concentrations of germinants may be selected such their addition to a population causes germination or does not cause germination.

Generally, the molecules that cause germination of a population of spores may be determined by testing. In some examples, various substances or combinations of substances may be added to a stable population of spores and a determination of whether germination occurs is made. In these tests, the environment in which the bacterial spores are placed may have an effect on the determination of germinants. For example, for a population of bacterial spores in a buffer that contains potassium, the testing may determine that a combination of L-alanine and D-glucose cause germination. However, if the same population of spores were in water, similar testing may determine that a combination of L-alanine, D-glucose, and KBr causes germination. Generally, there is at least some water present (e.g., an aqueous solution) for germination to occur. Herein, germinants are defined based on studies where spores are in water (nothing else added to the water) and candidate germinants are added to the mixture of spores in water. Assays of this type are described in Example 2 of this disclosure.

Some example molecules that may act as germinants, without limitation, include nutrient germinants or non-nutrient germinants. Example nutrient germinants may include amino acids, sugars, nucleosides, or salts. Example non-nutrient germinants may include lysozyme or other proteins, dodecylamine, calcium dipicolinate, and others.

Non-limiting examples of germinant molecules may include amino acids, salts, nucleosides, vitamins, and sugars. In some examples, amino acids may be L-amino acids. The amino acids may be classed in various ways. One method for classifying amino acids includes small amino acids (alanine, glycine), hydrophilic amino acids (cysteine, serine, threonine), hydrophobic amino acids (isoleucine, leucine, methionine, proline, valine), aromatic amino acids (phenylalanine, tryptophan, tyrosine), acidic amino acids (aspartic acid, glutamic acid), amide amino acids (asparagine, glutamine), and basic amino acids (arginine, histidine, lysine). Germinant molecules may include analogs of amino acids. Such analogs are known in the art.

In certain examples, some L-amino acids may be excluded from the subject matter encompassed by the term, germinants. In various examples, one or more of the following amino acids may be excluded: L-alanine, L-valine, L-proline, L-leucine, L-cysteine, L-threonine, L-glutamine, L-asparagine, or L-phenylalanine.

Example salts that are germinants, without limitation, may include KBr, KCl, MgSO₄, and NaCl. Non-limiting examples of purines/nucleosides may include adenine, adenosine, caffeine, guanine, guanosine, hypoxanthine, inosine, isoguanine, theobromine, uric acid, and xanthine. Non-limiting examples of vitamins may include β-alanine, biotin, folic acid, inositol, nicotinic acid, panthothenic acid, pyridoxine, riboflavin, and thiamine. Non-limiting examples of sugars may include arabinose, fructose, glucose, raffinose, and sucrose and lactose.

Non-limiting examples of germinants that may be suitable for the compositions, methods, and kits, described herein include lactate; lactose (as found in dairy products), bicarbonate or carbonate compounds such as sodium bicarbonate; carbon dioxide (e.g., carbonic acid: CO₂ dissolved in water, as is common in “sodas” or “soft drinks” such as cola or some fruit flavored beverages); compounds that adsorb lipid (e.g., starch, such as found in wheat, rice or other grains and potatoes and some other vegetables); charcoal or similar materials of high surface area that may adsorb or absorb fatty acid and lipid materials that may inhibit spore germination; monosaccharides such as fructose, glucose, mannose, or galactose; alanine, asparagine, cysteine, glutamine, norvatine, serine, threonine, valine, glycine, or other amino acid, and derivatives thereof such as N-(L-a-aspartyl)-L-phenylalanine (commonly sold under the trade name of “Aspartame”); inosine; bile salts such as taurocholate; and combinations of such spore germinants. For example, useful spore germinants can include alanine alone or in combination with lactate; a combination of L-asparagine, glucose, fructose, and potassium ion (AGFK); amino acids such as asparagine, cysteine, or serine alone or in combination with lactate; and caramels created by autoclaving monosaccharides or such caramels in combination with amino acids. In some examples, the composition comprises one or more germinants. In a particular embodiment, the composition comprises L-asparagine, glucose, fructose, and potassium ion (AGFK).

In this disclosure, water (i.e., H₂O) is not considered to be a germinant. That is, when the term “germinant” is used herein, water is excluded from the meaning of the term.

### Non-Germinants

Non-germinant molecules are molecules that do not have detectable germinant activity. Just as germinants may be identified through testing, non-germinants may be identified through similar testing. Generally, molecules may be defined as non-germinants based on lack of germinant activity in the testing. Herein, this testing is performed on a mixture of spores in water. Molecules to be tested for their germinant activity are added to the mixture of spores and water, and germination of the spores is determined. While it may not be possible to test a putative non-germinant molecule under every condition or circumstance that would reveal germinant activity, it generally is possible to specify a molecule as lacking germinant activity within the framework of the testing that has been performed. Again, the testing used in this disclosure was performed in water (see Example 2).

In some examples, testing of candidate substances for germinant activity or lack of germinant activity may be performed by adding the candidate substances to bacterial spores in water and determining whether the spores germinate into vegetative cells. The candidate molecules may be added to the spores at different amounts, in combination with other molecules, and the like. Absence of germinant activity may be reported within the framework of the particular testing performed.

In some examples, conclusions from testing used to determine that particular substances are non-germinants may be informed by how other substances perform in the same testing. In some examples, the framework for determining that a first molecule lacked germinant activity for particular spores in a specific assay may be accompanied by information that a second molecule displayed germinant activity for the same spores in the same assay. Non-germinant molecules that show no germinant activity under conditions in which other molecules show germinant activity may be called contextual non-germinants in this disclosure. That is, for a contextual non-germinant, the lack of germinant activity of the molecule in an assay is viewed within the context that one or more other molecules possess germinant activity in the same assay. Molecules shown to lack germinant activity, absent the context that other molecules show germinant activity under the same conditions, are not called contextual non-germinants in this disclosure.

Some examples of specific contexts that may be used to determine that molecules are contextual non-germinants are illustrated in the instances below.

In one example, L-alanine alone, at a concentration of 3 mM in water, causes germination of spores obtained from a specific bacterial strain, while 3 mM L-valine, under the same conditions, does not cause germination of the same spores. Under this information, L-valine may be said to be a contextual non-germinant for the tested spores.

In one example, 20 essential L-amino acids are separately tested, at concentrations of 3 mM in water, for germinant activity. L-alanine causes germination of the tested spores, while none of the other 19 L-amino acids, one being L-valine, causes germination of these spores. Under this information, L-valine may be said to be a contextual non-germinant for the tested spores.

In one example, 20 essential L-amino acids are separately tested, at concentrations of 3 mM in water, for germinant activity. L-alanine causes germination of the tested spores, as do all of the other L-amino acids tested, except for L-valine. Under this information, L-valine may be said to be a contextual non-germinant for the tested spores.

In the above examples, the molecules that cause spore germination and the molecules that do not cause spore germination are all amino acids. Therefore, in the above instances, the contextual non-germinants are the same type of molecules, or in the same group of molecules, as the germinants. Additional examples similar to this are illustrated in the examples below.

In one example, 3 mM of L-alanine, in combination with 3 mM of D-fructose, causes germination of a population of bacterial spores. L-cysteine, at 3 mM concentration, also causes germination of the spores in combination with 3 mM of D-fructose. L-valine, however, at 3 mM concentration, in combination with D-fructose, does not cause germination of the spores. Under this information, L-valine may be said to be a contextual non-germinant for the tested spores when in combination with D-fructose.

In one example, 3 mM of any one of L-alanine, L-histidine, L-isoleucine, L-leucine, L-phenylalanine, or L-proline, in combination with both 3 mM of KBr and 3 mM of D-fructose, causes germination of a population of bacterial spores. L-valine, at 3 mM concentration, in combination with 3 mM KBr and 3 mM D-fructose, does not cause germination of the spores. Under this information, L-valine may be said to be a contextual non-germinant for the tested spores when in combination with 3 mM KBr and 3 mM D-fructose.

These types of situations—where the molecules that are non-germinants are in the same group of molecules as the molecules that don't cause germination—may be useful, but may not be the only situation under which molecules can be determined to be contextual non-germinants. In other examples, the molecules that cause germination of spores may not be the same type of molecules, or in the same group of molecules, as the molecules that do not cause germination. An example of this is illustrated in the example below.

In one example, 3 mM of any one of L-alanine, L-histidine, L-isoleucine, L-leucine, L-phenylalanine, or L-proline, in combination with both 3 mM of KBr and 3 mM of D-fructose, causes germination of a population of bacterial spores. Riboflavin, herein classed in the vitamin group, at 3 mM concentration, does not cause germination of the spores in combination with 3 mM KBr and 3 mM D-fructose. Under this information, riboflavin may be considered a contextual non-germinant for the tested spores when in combination with 3 mM KBr and 3 mM D-fructose.

Herein, when it is said that a molecule has germinant activity or causes spores to germinant or is a germinant, this generally means that the molecule, alone or in combination with other germinants, results in at least some spores in a population of spores germinating into vegetative cells. In contrast, when it is said that a molecule does not have germinant activity or does not cause spores to germinate or is a non-germinant, this generally means that the molecule does not result in spores in a population of spores germinating into vegetative cells.

Differences between a germinant and a non-germinant may be relative, in some examples. For example, spores may germinate at a low level in presence of a non-germinant, but the proportion of spores that germinate in this population is less, in many cases much less, than the proportion of spores that germinate in a population in presence of a germinant. In various examples, the percentage of spores in a population that germinate in presence of sufficient germinants may be at least about 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, or 99 percentage points higher than the percentage of spores in a population that germinate in presence of a non-germinant (i.e., with no germinants present).

In some examples, non-germinant molecules may be the same types of molecules, or in the same groups of molecules as germinants. Non-limiting examples of non-germinants may include amino acids, salts, purines/nucleosides, vitamins, sugars, and other types of molecules.

In some examples, non-germinant amino acids may be L-amino acids. The amino acids may be classed in various ways. One method for classifying amino acids includes small amino acids (alanine, glycine), hydrophilic amino acids (cysteine, serine, threonine), hydrophobic amino acids (isoleucine, leucine, methionine, proline, valine), aromatic amino acids (phenylalanine, tryptophan, tyrosine), acidic amino acids (aspartic acid, glutamic acid), amide amino acids (asparagine, glutamine), and basic amino acids (arginine, histidine, lysine). Non-germinant molecules may include analogs of amino acids. In some examples, a non-germinant amino acid may be L-valine. In some examples, L-valine may be a non-germinant for Bacillus, Bacillus pumilus, or Bacillus pumilis strain SB3189. In some examples, a non-germinant amino acid may be L-asparagine. In some examples, L-asparagine may be a non-germinant for Bacillus, Bacillus megaterium, or Bacillus megaterium strain SB3112.

In certain examples, some L-amino acids may be excluded from the subject matter encompassed by the term, non-germinants. In various examples, one or more of the following amino acids may be excluded: L-alanine, L-valine, L-proline, L-leucine, L-cysteine, L-threonine, L-glutamine, L-asparagine, or L-phenylalanine.

Non-germinants may have to contact bacterial spores at certain concentrations to be considered non-germinants. In some examples, non-germinants may have to contact bacterial spores at certain concentrations to affect germination parameters (discussed below).

In some examples, non-germinants may be present in compositions disclosed herein at concentrations of between about 0.001 mM-10.0 M, 0.01 mM-5.0 M, 0.1 mM-1.0 M, or 1.0 mM-0.1 M. In some examples, the non-germinants may be present in compositions disclosed herein at concentrations of about 0.01, 0.05, 1, 2, 3, 4, 5, 6, 7, 8, 10, 15, 20, 25, 30, 35, 40, 45, 50, 60, 70, 80, 90, or 100 mM. Concentrations of non-germinants may be selected such their addition to a population causes changes in germination parameters as compared to spores germinated without non-germinants.

In this disclosure, water (i.e., H₂O) is not considered to be a non-germinant. That is, when the term “non-germinant” is used herein, water is excluded from the meaning of the term.

### Addition of Non-Germinants to Spores

In some examples, the non-germinants disclosed herein are used by initially adding them to spores and subsequently adding one or more germinants to the spores. That is, the non-germinants and the germinants may be sequentially added to spores. In some examples, the non-germinants disclosed herein are used by adding them to spores at the same time that one or more germinants are added to the spores. That is, the non-germinants and the germinants may be simultaneously added to spores.

In some examples, one or more non-germinants are initially added to a population of bacterial spores, with no germination of the spores occurring. This may be called pretreatment of the spores with the non-germinants. Subsequent to addition of the non-germinants to the spores, germinants sufficient to cause germination of the spores are added to the combination of the spores and the non-germinants. Generally, the spores germinate. Generally, one or more parameters of germination of the spores may be different than germination parameters of the same spores germinated in the same way, except without pretreatment with the non-germinants. In some examples, the different germination parameters may indicate that the spores germinate more efficiently (e.g., increased Gₘₐₓ, decreased T_{lag}) due to the pretreatment. The non-germinants may have to be present at certain amounts to affect germination parameters.

When one or more non-germinants are added to bacterial spores initially, these may be in contact with the spores for various periods of time before germinants are subsequently added to cause germination of the spores. In various examples, the non-germinants added initially may be in contact with the spores for about 1, 2, 3, 4, 5, 10, 15, 20, or 30 minutes; about 1, 2, 3, 4, 5, 6, 8, 10, 12, 15, or 18 hours; about 1, 2, 3, 4, 5, 10, 15, or 20 days; about 1, 2, 3, 4, 5, 6, 8, or 10 months; or about 1, 2, 3, 4, or 5 years; before subsequent germinants are added to cause germination of the spores.

Likewise, the duration between the initial addition of non-germinants and the subsequent addition of germinants may vary. In various examples, the subsequent addition or contacting of a population of bacterial spores with germinants may occur about 1, 2, 3, 4, 5, 10, 15, 20, or 30 minutes after; about 1, 2, 3, 4, 5, 6, 8, 10, 12, 15, or 18 hours after; about 1, 2, 3, 4, 5, 10, 15, or 20 days after, about 1, 2, 3, 4, 5, 6, 8, or 10 months after; or about 1, 2, 3, 4, or 5 years after the initial addition or contacting of the population of bacterial spores with non-germinants.

In some examples, bacterial spores and the one or more non-germinants initially added to the spores may be kept at various temperatures for at least part of the time before subsequent germinants are added to cause germination of the spores. In various examples, the temperature may be 4° C., 5° C., 8° C., 10° C., 15° C., 20° C., 25° C., 30° C., 37° C., 42° C., 50° C., 60° C., 70° C., 80° C., or 90° C.

While a single “initial” addition of one or more non-germinants to bacterial spores is generally referred to, it may be that multiple “initial” additions of non-germinants to the bacterial spores may be used. These initial additions occur before the subsequent addition of germinants to the spores. The subsequent addition generally causes germination of the spores. Generally, we have found that, in order to affect parameters of germination, the non-germinants need to be present with the spores at the time the germinants are added.

Water is generally present in order for germination of the spores to occur. In an example where a population of bacterial spores and a non-germinant is present in an aqueous solution, subsequent addition of the germinants may cause germination of the spores. However, where a population of bacterial spores and a non-germinant is present in a non-aqueous form, subsequent addition of the germinant, absent water, may not cause germination. Generally, water is present with the spores, non-germinants, and the germinants in order for germination of the spores to occur.

Also disclosed herein are compositions of bacterial spores and a non-germinant (e.g., bacterial spores and the non-germinants added initially, as described above). The bacterial spores in these compositions are stable in that they do not germinate until sufficient germinants are added to the compositions (e.g., subsequent addition of germinants, as described above). These compositions may be, without limitation, in a solid state or a liquid state. In some examples, solid compositions may be made, without limitation, using techniques like spray drying, freeze drying, air drying, or drum drying. The solid compositions may be dry. In some examples, dry compositions may have a moisture content of less than about 50%, 40%, 30%, 25%, 20%, 15%, 12%, 10%, 9%, 8%, 7%, 6%, 5%, 4%, 3%, 2%, or 1%.

Regardless of the state of the compositions, the spores in these compositions are stable in that they are generally not germinating. However, they can germinate when provided with sufficient germinants (generally water is also needed). There do exist in the art, stable dry compositions of bacterial spores that contain full complements of germinants (in one example, called “intimate mixtures” of spores and germinants). When water is added to these intimate mixture compositions, the bacterial spores in the compositions germinate. The solid compositions described herein—stable bacterial spores that contain non-germinants—which may be dry, generally do not germinate when water is added, because a full complement of germinants is not present.

Also disclosed herein are compositions of bacterial spores, a non-germinant, and a partial complement of germinants. The spores in these compositions are stable and not germinating. The compositions may be dry or liquid. These compositions do not contain a full complement of germinants. For a liquid composition, addition of sufficient germinants can cause germination. For a dry composition, addition of sufficient germinants and water can cause germination.

Also disclosed herein are dry compositions of bacterial spores, a non-germinant, and sufficient germinants. Such compositions have a moisture content below the level needed for the spores in the composition to germinate. Therefore, the spores in such compositions are stable, in that they are not germinating. The spores in these compositions do germinate when water is added. These compositions—containing spores, a non-germinant, and germinants sufficient for germination—are different from the “intimate mixtures” of spores and germinants described above. The so-called intimate mixtures do not contain known non-germinants that affect parameters of germination when the spores are caused to germinate.

Also disclosed herein are liquid compositions of bacterial spores, a non-germinant, and sufficient germinants. Such compositions may only exist for a time after water is added to dry compositions of bacterial spores, non-germinants, and sufficient germinants. Generally, when water is added to such compositions, the spores germinate.

Generally, the compositions described herein that contain bacterial spores and non-germinants, and may contain additional substances, can be said to have enhanced properties because they may germinate with different germination parameters than compositions that do not have non-germinants. These compositions, containing bacterial spores and non-germinants, are significantly more than the individual spores and individual non-germinants. When combined, the spores have the potential to germinate in a way that they could not without the non-germinants.

### Effects of Non-Germinants on Germination Parameters

This disclosure concerns non-germinants that affect one or more parameters of spore germination. That is, the presence of a non-germinant in a population of spores can affect parameters of germination when the spores germinate in response to sufficient germinants. In some instances, pretreatment of spores with the non-germinants, or simultaneous addition of the non-germinants with sufficient germinants, may result in more efficient germination of the spores, as compared to germination in absence of the non-germinants. Non-germinants that make spores germinate more efficiently may be said to enhance germination of spores. Compositions of bacterial spores that contain these non-germinants may be said to have enhanced properties.

In some instances, pretreatment of spores with certain non-germinants, or simultaneous addition to spores of certain non-germinants and sufficient germinants, may result in less efficient germination of the spores, as compared to germination in absence of the non-germinants. While this disclosure may encompass certain non-germinants that produce this result, certain non-germinant molecules of this type are generally excluded from the scope of this disclosure. In some examples, a non-germinant that is toxic to bacterial spores or kills bacterial spores could be considered a non-germinant that results in less efficient germination. However, these types of molecules are generally excluded from the scope of this disclosure. In some examples, there are molecules already known in the art that inhibit spore germination. Known inhibitors of germination are generally excluded from the scope of this disclosure. Some known inhibitors of germination may be alkyl alcohols, phenols, organic acids, ion-channel blockers, protease inhibitors, sulphydryl reagents, calmodulin antagonists, azide, theophylline, potassium sorbate, and other molecules. Some of the known inhibitors of germination may be toxic to or kill spores. Some molecules that are toxic to or kill spores may be known inhibitors of germination.

To determine whether a non-germinant affects germination, a variety of parameters of a germinating spore population can be measured. T_{lag}, Gₘₐₓ, Gᵣₐₜₑ, and germination heterogeneity are example parameters, but not the only parameters, of a germinating spore population that can be measured. The meaning of these particular terms and examples of these parameters may be determined can be found in the Definitions section and in FIG. 1 of this disclosure. In general, a first population of bacterial spores that has a decreased T_{lag}, increased Gₘₐₓ, increased Gᵣₐₜₑ, or decreased germination heterogeneity, as compared to a second population of bacterial spores, may be said to germinate more efficiently than the second population of bacterial spores.

In this disclosure, when it is said that a non-germinant results in germination of a population of spores that is different than germination of a population of spores without the non-germinant, the differences may be determined by comparing like germination parameters for germination of the two spore populations. As discussed, some germination parameters that may be determined and compared may include T_{lag}, Gₘₐₓ, Gᵣₐₜₑ, and germination heterogeneity. In some examples, the spore population pretreated with a non-germinant may have germination parameters that indicate more efficient germination than the spore population not treated with the non-germinant. For example, the T_{lag} for an efficiently germinating spore population may be less than the T_{lag} for a less efficiently germination spore population. The Gₘₐₓ for an efficiently germinating spore population may be greater than the Gₘₐₓ for a less efficiently germinating spore population. The Gᵣₐₜₑ for an efficiently germinating spore population may be greater than the Gᵣₐₜₑ for a less efficiently germinating spore population. The germination heterogeneity for an efficiently germinating spore population may be less than the germination heterogeneity for a less efficiently germinating spore population. For a given germination parameter, these differences between values for an efficiently germinating spore population (e.g., with non-germinants) as compared to a less efficiently germinating spore population (e.g., without non-germinants) may be at least about 1%, 2%, 3%, 4%, 5%, 6%, 7%, 8%, 9%, 10%, 12%, 14%, 16%, 18%, 20%, 25%, 30%, 35%, 40%, 45%, 50%, 60%, 70%, 80%, 90%, 100%, 200%, 300%, 400%, 500%, 600%, 700%, 800%, 900%, or 1000%, depending on the units used for measuring specific germination parameters.

Pretreatment of spores with a non-germinant may cause germination that has one or more of these parameters of increased germination efficiency, as compared to germination of the same spores without pretreatment with non-germinants. Simultaneous addition of a non-germinant to spores in combination with sufficient germinants may also result in germination with one or more parameters of increased germination efficiency, as compared to addition of sufficient germinants absent the non-germinant. When a germination parameter is affected by a non-germinant and indicates more efficient germination, other parameters may not be affected, or may be affected in a way indicating less efficient germination. That is, non-germinant effects on germination may not affect all measurable germination parameters or may not affect all measurable germination parameters in the same way. In some examples, a population of spores may be said to germinate more efficiently if changes to one germination parameter are consistent with more efficient germination. The other parameters may be unchanged or, in some examples, may change in a way suggesting less efficient germination, at least for that parameter. In some examples, a non-germinant may be selected based on the germination parameter that is desired to be affected. For example, it may be important to use a non-germinant that increases the Gₘₐₓ of a population of germinating spores. The fact that T_{lag} may also be increased by the non-germinant may not be a major concern in selection of the non-germinant.

As already described, the non-germinants that are the subject of this disclosure are those that affect one or more parameters of spore germination. In some examples, one or more affected germination parameters may indicate more efficient germination of spores. Therefore, the molecules disclosed herein have at least two properties. The first property is that the molecules are non-germinants (they generally lack the ability to cause at least some bacterial spores to germinate). The second property is that the molecules affect one or more germination parameters. In some examples, the molecules disclosed herein may first be determined to be non-germinants (e.g., using example studies as in Example 2), and then be determined to affect parameters of spore germination (e.g., using example studies as in Examples 3 and 4). In some examples, the molecules disclosed herein may first be determined to affect parameters of spore germination, and then be determined to be non-germinants.

In some examples, the disclosed non-germinants, in combination with germinants sufficient to cause germination, can be said to have synergistic effects on germination of a population of spores. Synergy occurs when the effect of a combination of things (e.g., molecules) on an outcome (e.g., germination) is greater than the sum of the individual members of the combination on an outcome. Herein, when the germinants alone are added to a population of spores, germination occurs and values for germination parameters (e.g., T_{lag}, Gₘₐₓ, Gᵣₐₜₑ, germination heterogeneity) can be obtained. When the non-germinants alone are added to a population of spores, no germination occurs and no values for germination parameters can be obtained—the effect of the non-germinants alone is zero. However, when the non-germinants are used in combination with the germinants, the effect on germination parameters is better than the sum of the effects of the non-germinants alone and the germinants alone—there is synergy.

In some examples, the non-germinants disclosed herein are said to be “known” to lack germinant activity on specific spores. In some examples, the disclosed non-germinants are said to be “known” to affect germination parameters of a population of spores (e.g., to cause more efficient germination of the spores). Generally, when the word “known” is used in these contexts in claims to a composition, for example, it means that there was knowledge that the claimed non-germinants lacked germinant activity on spores in the composition, or would affect germination parameters of the spores in the composition when germinated. Generally, when the word “known” is used in these contexts in claims to a method, for example, it means that there was knowledge that the non-germinants used in the claimed method lacked germinant activity on spores used in the method, or would affect germination parameters of the spores used in the claimed method. Use of “known” in this way in the claims is generally a limitation and means that prior art compositions or methods, practiced without this knowledge, is not prior art that supports a valid claim rejection.

### Uses of Non-Germinants

The compositions, methods, and kits disclosed herein may be used in a variety of circumstances. In some examples, a composition may be designed to contain bacterial spores because spores are known to be tolerant to a variety of adverse environmental conditions. Such a composition may have a longer shelf life and/or may better survive environmental insults than a product containing vegetative bacteria. When these compositions are subsequently used, by adding the compositions to an environment for example, the spores may need to germinate to have the intended effect on or in the environment. Therefore, it may be of interest to use the compositions, methods, or kits described herein, to achieve efficient germination of the spores.

In some examples, the compositions, methods, and kits disclosed herein may be used for bacterial spores applied to plants and/or plant leaves (i.e., agricultural use). In some examples, when so applied, the spores may germinate to vegetative bacterial cells which provide a useful function to plants. In some examples, the bacteria may provide biocontrol properties to the plant and/or enhance plant growth.

In some examples, the compositions, methods, and kits disclosed herein may be used in animal feed. Example bacterial spore-containing compositions may be mixed with animal feed or animal feed ingredients. This may be referred to as mash feed. In some examples, germination of the bacterial spores to vegetative bacteria in the mash feed may facilitate chemical breakdown of components of the mash. This may facilitate digestion of the mash feed in the animal digestive system or otherwise improve the digestive system of the animal.

In some examples, the compositions, methods, and kits disclosed herein may be used in detergents. The bacteria that produce the spores may be selected for inclusion in a detergent based on their ability to produce enzymes that may digest, for example, stains in a fabric. In some examples, deployment of the detergent may result in germination of the bacterial spores therein and production of the desired enzymes by the vegetative bacteria.

### Example Embodiments of the Invention

1. A first composition, comprising, consisting essentially of, or consisting of:
a stable population of bacterial spores in contact with a non-germinant;
the population of bacterial spores in the first composition known to be able to germinate with a different parameter as compared to germination of the population of bacterial spores in a second composition that does not contain the non-germinant.

2. The first composition of embodiment 1, where the non-germinant is a contextual non-germinant.

3. The first composition of any one of embodiments 1 or 2, where the non-germinant is a first L-amino acid.

4. The first composition of embodiment 3, where the first L-amino acid is a contextual non-germinant where a second L-amino acid is a germinant for the population of bacterial spores.

5. The first composition of any one of embodiments 3 or 4, where the first L-amino acid includes L-valine or L-asparagine.

6. The first composition of any one of embodiments 1-5, where the different parameter includes T_{lag}, germination heterogeneity, Gₘₐₓ, or Gᵣₐₜₑ.

7 The first composition of any one of embodiments 1-6, where the different parameter in the first composition is a decreased T_{lag}, decreased germination heterogentity, increased Gₘₐₓ, or increased Gᵣₐₜₑ, as compared to the second composition.

8. The first composition of any one of embodiments 1-7, where the first composition is dry.

9. The first composition of embodiments 8, where the dry composition does not germinate when water is added to the composition.

10. The first composition of any one of embodiments 1-9, where moisture content of the first composition is less than about is less than about 50%, 40%, 30%, 25%, 20%, 15%, 12%, 10%, 9%, 8%, 7%, 6%, 5%, 4%, 3%, 2%, or 1%.

11. The first composition of any one of embodiment 1-7, where the composition is a liquid.

12. The first composition of any one of embodiments 1-11, where the first composition includes a partial complement of germinants.

13. The first composition of any one of embodiments 1-12, where the non-germinant is defined using as assay of bacterial spores in water.

14. The first composition of any one of embodiments 1-13, where non-germinants do not include molecules toxic to the bacterial spores or that kill the bacterial spores.

15. The first composition of any one of embodiments 1-14, where non-germinant molecules do not include known inhibitors of germination.

16. The first composition of any one of embodiments 1-15, where the different parameter indicates more efficient germination of the bacterial spores in the first composition.

17. A first composition, comprising, consisting essentially of, or consisting of:
a population of bacterial spores and a non-germinant contacting the bacterial spores;
the population of bacterial spores in the first composition able to germinate when contacted with sufficient germinants in water;
where, when the population of bacterial spores in the first composition is contacted with the sufficient germinants, and germination of the population of bacterial spores occurs, a parameter of the germination is different as compared to germination of the population of bacterial spores in a second composition that does not contain the non-germinant when contacted with the sufficient germinants.

18. The first composition of embodiment 17, where the parameter of the germination that is different indicates more efficient germination of the population of bacterial spores in the first composition.

19. The first composition of any one of embodiments 17 or 18, where the non-germinant excludes molecules toxic to, or capable of killing, the population of bacterial spores, and excludes known inhibitors of germination.

20. The first composition of any one of embodiments 17-19, where the non-germinant is a contextual non-germinant.

21. The first composition of any one of embodiments 17-20, where the different parameter in the first composition is a decreased T_{lag}, decreased germination heterogeneity, increased Gₘₐₓ, or increased Gᵣₐₜₑ, as compared to the second composition.

22. The first composition of any one of embodiments 17-21, where the first composition includes a partial complement of germinants.

23. The first composition of any one of embodiments 17-20, where the first composition includes a full complement of germinants and is dry.

24. The first composition of any one of embodiments 17-20, where the first composition includes a full complement of germinants and is a liquid.

25. A composition comprising, consisting essentially of, or consisting of:
a population of bacterial spores in contact with one or more substances that do not have germinant activity on the bacterial spores when in water;
the one or more substances known to contribute to more efficient germination of the population of bacterial spores, when sufficient germinants in water are added to the composition, as compared to germination of the population of spores that do not contain the one or more first substances when sufficient germinants in water are added.

26. The composition of embodiment 25, where at least one of the substances is a contextual non-germinant.

27. The composition of any one of embodiments 25 or 26, where the one or more substances include an L-amino acid, salt, purine or nucleoside, vitamin, or sugar.

28. The composition of any one of embodiments 25-27, where the one or more substances includes a first L-amino acid that is a contextual non-germinant where a second amino acid is a germinant for the population of bacterial spores.

29. The composition of embodiment 28, where the first L-amino acid includes L-valine or L-asparagine.

30. The composition of any one of embodiments 25-29, where more efficient germination means that the population of bacterial spores in the composition in contact with the one or more substances germinates with a decreased T_{lag}, decreased germination heterogeneity, increased Gₘₐₓ, or increased Gᵣₐₜₑ, as compared to the population of bacterial spores in a composition without the one or more substances.

31. The composition of any one of embodiments 25-30, where more efficient germination means that the population of bacterial spores in the composition in contact with the one or more substances germinates with an increased Gₘₐₓ as compared to the population of bacterial spores in the composition without the one or more substances.

32. The composition of any one of embodiments 25-31, where the composition is dry.

33. The composition of any one of embodiments 25-32, where moisture content of the composition is less than about than about 50%, 40%, 30%, 25%, 20%, 15%, 12%, 10%, 9%, 8%, 7%, 6%, 5%, 4%, 3%, 2%, or 1%.

34. The composition of any one of embodiments 25-31, where the composition is a liquid.

35. The composition of any one of embodiments 25-34, where the bacterial spores are from bacteria from the genera Acetonema, Actinomyces, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Coxiella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halobacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, or Seinonella.

36. The composition of any one of embodiments 25-35, where the bacterial spores are from the bacteria Bacillus amyloliquefaciens, Bacillus pumilis, Bacillus megaterium, or Bacillus subtilis.

37. The composition of any one of embodiments 25-36, where the bacterial spores are heat activated.

38. The composition of any one of embodiments 25-37, where the one or more substances are present at a concentration of at least about 0.01, 0.05, 1, 2, 3, 4, 5, 6, 7, 8, 10, 15, 20, 25, 30, 35, 40, 45, 50, 60, 70, 80, 90, or 100 mM.

39. The composition of any one of embodiments 25-38, where the one or more first substances is one substance.

40. The composition of any one of embodiments 25-39, where the population of bacterial spores contains less than about 1%, 2%, 3%, 4%, 5%, 6%, 7%, 8%, 9%, 10%, 15%, 20%, 25%, 30%, 40%, or 50% vegetative cells.

41. The composition of any one of embodiments 25-40, where a percentage of the population of bacterial spores that germinates with sufficient germinants in water is at least about 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, or 99 percentage points higher than a percentage of the population of bacterial spores that germinates without the one or more substances, with the sufficient germinants in water.

42. The composition of any one of embodiments 25-41, where the composition includes a partial complement of germinants.

43. A first composition, comprising, consisting essentially of, or consisting of:
a stable population of bacterial spores;
a known non-germinant for the population of bacterial spores; and
a full complement of germinants for the bacterial spores;
where the composition is dry;
where the known non-germinant and the full complement of germinants are in contact with the bacterial spores in the population; and
where the population of bacterial spores in the first composition germinates more efficiently when water is added, compared to germination of the bacterial spores in a second dry composition that includes the full complement of germinants in contact with the bacterial spores but does not contain the known non-germinant.

44. The first composition of embodiment 43, where moisture content of the first composition is less than about 50%, 40%, 30%, 25%, 20%, 15%, 12%, 10%, 9%, 8%, 7%, 6%, 5%, 4%, 3%, 2%, or 1%.

45. A method, comprising, consisting essentially of, or consisting of:
contacting a population of bacterial spores with amounts of one or more non-germinants for the bacterial spores;
the amounts of the one or more non-germinants known to contribute to more efficient germination of the population of bacterial spores, when sufficient germinants are added in water, as compared to germination of the population of bacterial spores, without the one or more non-germinants, when sufficient germinants are added in water;
the population of bacterial spores and the one or more non-germinants forming a mixture.

46. The method of embodiment 45, where at least one of the non-germinants is a contextual non-germinant.

47. The method of any one of embodiments 45 or 46, where at least one of the non-germinants includes an L-amino acid, salt, purine or nucleoside, vitamin, or sugar.

48. The method of any one of embodiments 45-47, where at least one of the non-germinants includes a first L-amino acid which is a contextual non-germinant and where a second amino acid is a germinant for the population of bacterial spores.

49. The method of any one of embodiments 45-48, where at least one of the non-germinants includes L-valine or L-asparagine.

50. The method of any one of embodiments 45-49, where more efficient germination means that the population of bacterial spores in the composition in contact with the one or more substances germinates with a decreased T_{lag}, decreased germination heterogeneity, increased Gₘₐₓ, or increased Gᵣₐₜₑ, as compared to the population of bacterial spores in a composition not in contact with the one or more substances.

51. The method of any one of embodiments 45-50, where more efficient germination means that the population of bacterial spores in the composition in contact with the one or more substances germinates with an increased Gₘₐₓ as compared to the population of bacterial spores in the composition without the one or more substances.

52. The method of any one of embodiments 45-51, where the mixture is dry.

53. The method of embodiments 45-52 where moisture content of the mixture is less than about 50%, 40%, 30%, 25%, 20%, 15%, 12%, 10%, 9%, 8%, 7%, 6%, 5%, 4%, 3%, 2%, or 1%.

54. The method of any one of embodiments 45-51, where the mixture is a liquid.

55. The method of any one of embodiments 45-54, where the amounts of the one or more non-germinants in the mixture are between about 0.001 mM-10.0 M, 0.01 mM-5.0 M, 0.1 mM-1.0 M, or 1.0 mM-0.1 M.

56. The method of any one of embodiments 45-54, where the amounts of the one or more non-germinants in the mixture are at least about 0.01, 0.05, 1, 2, 3, 4, 5, 6, 7, 8, 10, 15, 20, 25, 30, 35, 40, 45, 50, 60, 70, 80, 90, or 100 mM.

57. The method of any one of embodiments 45-56, where the population of bacterial spores is from the bacteria Bacillus amyloliquefaciens, Bacillus pumilis, Bacillus megaterium, or Bacillus subtilis.

58. The method of any one of embodiments 45-57, where the population of bacterial spores is heat activated.

59. The method of any one of embodiments 45-58, including contacting the population of bacterial spores in the mixture with a partial complement of germinants.

60. The method of any one of embodiments 45-59, including contacting the population of bacterial spores in the mixture with a full complement of germinants.

61. The method of any one of embodiments 45-60, including dispersing the mixture into an environment.

62. The mixture of any one of embodiments 45-61.

63. A kit, comprising, consisting essentially of, or consisting of:
a population of stable bacterial spores; and
a known non-germinant for the population of stable bacterial spores that, when contacted with the bacterial spores is known to contribute to more efficient germination of the bacterial spores.

64. The kit of embodiment 63, including a partial complement of germinants for the population of stable bacterial spores.

65. The kit of any one of embodiments 63 or 64, including a full complement of germinants for the population of stable bacterial spores

66. A composition, comprising, consisting essentially of, or consisting of:
a population of bacterial spores, and a non-germinant in an amount sufficient to synergistically enhance at least one germination parameter when the population of bacterial spores is contacted with a full complement of germinants.

67. A method, comprising, consisting essentially of, or consisting of:
contacting a population of bacterial spores, with a non-germinant in an amount sufficient to synergistically enhance at least one germination parameter when the population of bacterial spores is contacted with a full complement of germinants.

### EXAMPLES

The following examples are for the purpose of illustrating various embodiments and are not to be construed as limitations.

### Example 1. Preparation of Bacterial Spores

Bacteria from which spores were to be prepared were grown logarithmically in liquid culture. As carbon, nitrogen, and/or phosphorus in the logarithmic cultures became limiting (e.g., late in logarithmic growth), the vegetative cells began to sporulate. The cultures continued to be incubated until it was estimated that no additional spores would form in the cultures. In some cases, the spores were obtained from cultures that were production runs. The cultures were then centrifuged to pellet the spores, and remaining cells and debris. When these spore pellets were suspended in water, washed, again suspended in water, and the spore suspension allowed to settle in a tube, three visible layers generally formed. Microscopic examination of samples was used to confirm the presence of phase-bright spores at a desired purity (>99% phase-bright spores). If purity was not achieved, then water washing was repeated until desired purity was reached.

For the experiments described in Example 2, spores were prepared by subjecting the upper layer of settled spores to HistoDenz™ density gradient centrifugation. A 10-15 ml aliquot of the upper layer of settled spores was mixed with 20 ml of water in a tube. Larger cellular debris sedimented to the bottom of the tube while spores generally remained suspended in the water. The water containing the spores was transferred to a separate tube, while the cellular debris was left behind. The spores were centrifuged in a clinical centrifuge for 5 min at 8,000 rpm. The supernatant was discarded and the pellet was suspended in 25 ml of deionized water. After two additional washing steps, 10 ml of the spores were diluted with 20 ml of ice cold autoclaved water, and centrifuged for 5 min at 15,000 rpm. The spores formed a pellet which was suspended in ice cold autoclaved water. This centrifugation and suspension step was repeated two additional times. The spore pellet was then suspended in 5 ml of 20% HistoDenz™ (Sigma-Aldrich) in water (1 g HistoDenz™ in 5 ml of water). The spores in HistoDenz™ were layered on top of a 50% HistoDenz™ solution (5 g HistoDenz™ in 10 ml of water) in a 50 ml centrifuge tube. This tube was centrifuged for 35 min at 11,500 rpm. The bacterial spores formed a pellet at the bottom of the tube (vegetative cells and cellular debris formed a layer within the HistoDenz™ solution). The pellet was suspended in 5 ml of autoclaved water, diluted to a final volume of 50 ml and stored at 4° C. until needed.

### Example 2. Identifying Germinants and Nongerminants

Studies were performed to determine chemical/molecular requirements for germination of spores from various bacterial strains. Various amino acids, purines, nucleosides, sugars, salts, and vitamins were added to spores in water to determine whether these substances caused germination of the spores. The strategy was to first prepare and test combinations of these potential germinants. If a particular combination caused germination, subsequent combinations that contained a subset of the original combination were tested. Using this approach iteratively, it was generally possible to identify some requirements for germination. Molecules that did not have germinant activity in the assays could be considered non-germinants.

For example, a first combination of potential germinants containing 3 mM of all 20 essential L-amino acids was tested for ability to cause germination. If this combination caused germination, we concluded that one or more of the individual amino acids of the combination were required. Additional experiments were then performed using a second combination of amino acids, where one or more amino acids present in the first combination had been omitted (i.e., the second combination was a subset of the first combination). For example, amino acid solutions that omitted one or more specific of the amino acid subgroups indicated in Table 1 (e.g., small, hydrophilic, hydrophobic, aromatic, acidic, amide, basic) were prepared. If the spores germinated with the first combination, but did not germinate with the second combination, we concluded that one or more of the omitted components was needed. Using this approach, we were able to ascertain some minimal germination requirements for bacterial spores from a variety of strains.

TABLE 1
Substances tested for germinant activity

| Substance | Substance | |
|---|---|---|
| group | subgroup | Substance |
| Amino acids¹ | Small | Alanine, Glycine |
| | Hydrophilic | Cysteine, Serine, Threonine |
| | Hydrophobic | Isoleucine, Leucine, Methionine, Proline, |
| | | Valine |
| | Aromatic | Phenylalanine, Tryptophan, Tyrosine |
| | Acidic | Aspartic acid, Glutamic acid |
| | Amide | Asparagine, Glutamine |
| | Basic | Arginine, Histidine, Lysine |
| Salts | — | KBr, KCl, MgSO₄, NaCl |
| Purines/ | — | Adenine, Adenosine, Caffeine, Guanine, |
| nucleosides | | Guanosine, Hypoxanthine, Inosine, |
| | | Isoguanine, Theobromine, Uric acid, |
| | | Xanthine |
| Vitamins | — | β-alanine, Biotin, Folic acid, Inositol, |
| | | Nicotinic acid, Panthothenic acid, |
| | | Pyridoxine, Riboflavin, Thiamine |
| Sugars | — | Arabinose, D-fructose, glucose, raffinose, |
| | | sucrose, lactose |

¹Amino acids were L amino acids

To perform these studies, spores prepared as described in Example 1 were first heat activated in water at 68° C. for 30 min, cooled to room temperature, and suspended in 100 mM sodium phosphate buffer, pH 7.0, such that optical density of the suspension at 580 nm was 1.0 (OD₅₈₀=1.0). The heat-activated spores were checked for auto-germination by monitoring changes in light refraction for 20 min at 580 nm using a Tecan Infinite M200 96-well plate reader. Decreases in light refraction indicated spore germination occurred.

Spore preparations that did not auto-germinate were tested for ability to germinate under conditions where many substances with possible germinant activity were present by suspending the spores in brain heart infusion medium (BHI), a relatively rich medium. Spores that germinated in BHI medium were used in further experiments to determine specific germinant requirements.

The germinant testing experiments were performed in 96-well plates in a 200 μl final well volume (60 μl of the heat-activated spore stock in sodium phosphate buffer with OD₅₈₀=1.0, and 140 μl of germinant solution in water). Each potential germinant substance was present at a final concentration of 3 mM. The reaction mixtures were incubated for 70 minutes at 37° C. and OD₅₈₀ measurements were taken every minute during that time (decreased OD indicated germination). The readings were normalized by dividing each OD₅₈₀ reading by the reading obtained at time 0 (i.e., the time at which the germinant solution was added to the spores) to give relative OD₅₈₀ readings. Generally, germination of spore preparations, as shown by OD₅₈₀ readings, was confirmed using phase contrast microscopy and/or malachite green staining with a safranin counterstain (i.e., Schaeffer-Fulton method).

In the initial experiment for each spore preparation, the germinant solution contained all of the substances shown in Table 1 at a concentration of 3 mM (i.e., complete defined medium). If the spores germinated under these conditions, then subsequent iterative experiments used germinant solutions that lacked specific groups of germinants, as described above, to ascertain specific minimal germinant requirements for spores from a specific bacterial strain.

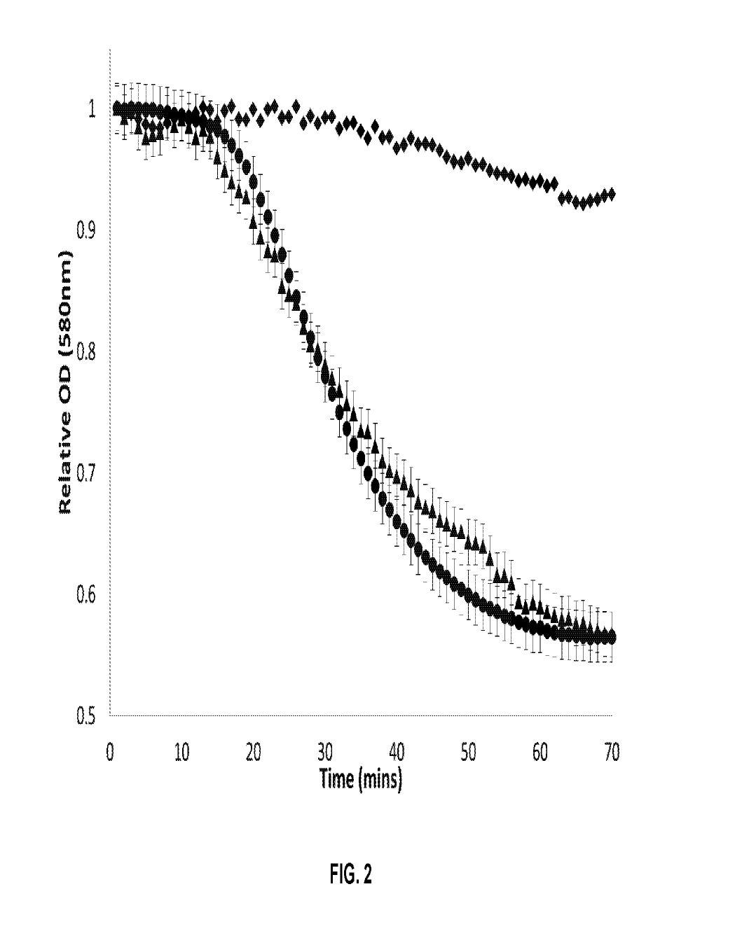

In some examples, spores from Bacillus amyloliquefaciens strain SB3615 were used in these experiments. SB3615 spores germinated in BHI medium (FIG. 2) and in complete defined medium (which contained all substances listed in Table 1 at 3 mM). Elimination of sugars, salts, vitamins, and purines/nucleosides had no effect on germination rates (i.e., these spores germinated in the presence of all 20 essential L-amino acids, without any other substances), which indicated that one or more amino acids likely caused germination of these spores. Therefore, we did germination experiments with each of the 20 essential L-amino acids alone. FIG. 2 shows data from one of these experiments, using L-alanine alone (3 mM concentration). The data indicate that the spores essentially germinated as well in the presence of L-alanine as they did in BHI medium (positive control). The spores did not germinate when no germinants were used (negative, buffer control). These data showed, that for spores from the SB3615 strain, that 3 mM L-alanine alone could cause germination.

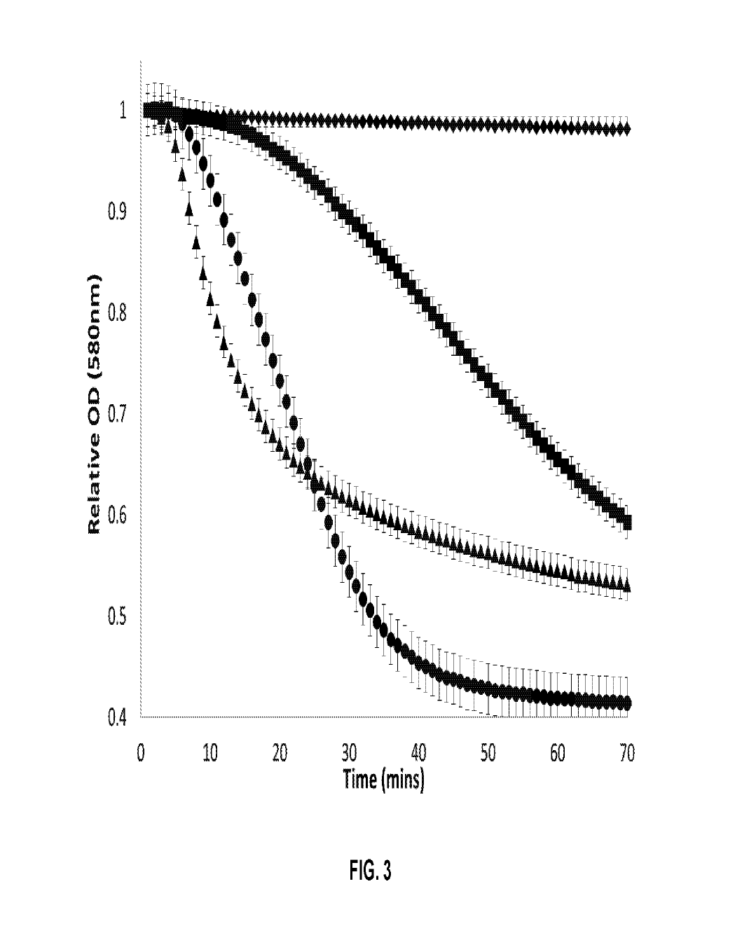

In another example, spores from Bacillus pumilus strain SB3189 were used in germination experiments. SB3189 spores germinated in BHI medium (FIG. 3) and in complete defined medium. Elimination of vitamins and purines/nucleosides had no effect on germination. These spores also germinated in a mixture of all 20 essential L-amino acids, sugars, and salts listed in Table 1. We tested each individual amino acid with individual sugars, and each individual amino acid with salts. From these experiments, we determined that combinations of L-alanine and D-fructose, or L-cysteine and D-fructose, all at 3 mM concentration, were sufficient to cause germination of these spores, as shown in FIG. 3.

We also found that sucrose could substitute for fructose, in combination with either L-alanine or L-cysteine. One possibility to explain the sucrose substitution was that sucrose degraded to glucose and fructose, and the released fructose fulfilled the germination requirement. We did not investigate this possibility. L-alanine in combination with either fructose or sucrose caused faster germination (lower T_{lag}) than did L-cysteine in combination with either fructose or sucrose. None of L-alanine, L-cysteine, D-fructose, or sucrose alone, at a concentration of 3 mM, caused germination.

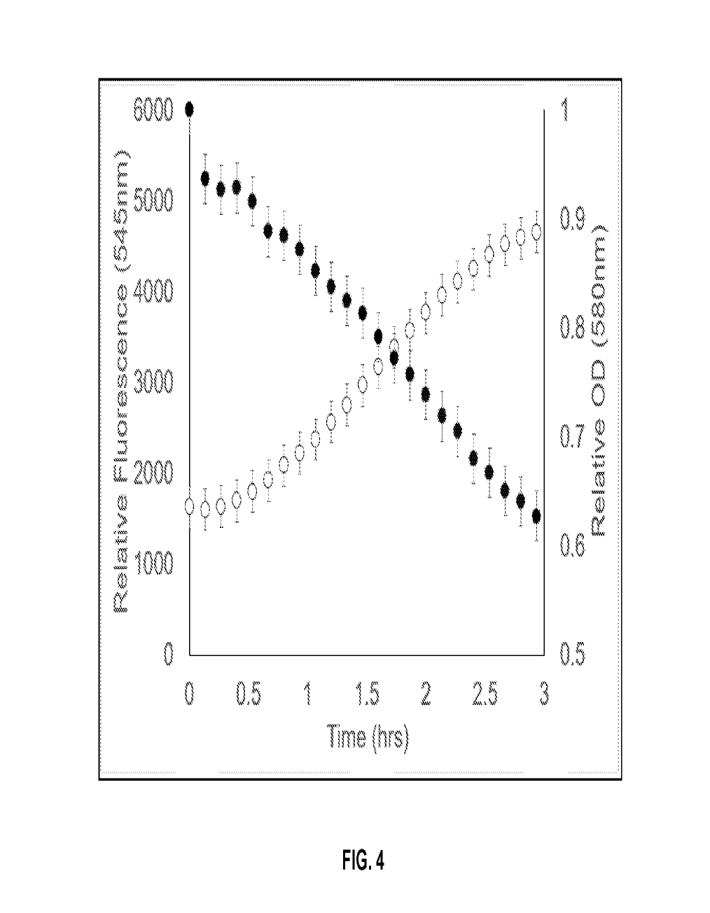

In another example, spores from Bacillus megaterium strain SB3112 were used in germination experiments. SB3112 spores germinated in complete defined medium. Elimination of vitamins had no effect on germination. Since these spores could germinate in a mixture of L-amino acids, purines/nucleosides, sugars, and salts, as shown in Table 1, we tested each group (i.e., amino acids, purines/nucleosides, sugars, salts) individually and in combinations of two of the groups, three or the groups, and four of the groups. From these experiments, we found that the six amino acids listed in Table 2, along with KBr and D-glucose, caused germination of the spores. Germination for the combination of L-proline, KBr, and D-glucose is shown in FIG. 4.

The germination requirements for a variety of bacterial strains were determined using similar experiments. Table 2 shows the conclusions from these studies.

TABLE 2
Minimal germinant requirements for various bacteria

| Genus and species of spores | Strain | Minimal germinant requirement¹ |
|---|---|---|
| Bacillus amyloliquefaciens | SB3615 | L-alanine |
| Bacillus pumilus | SB3189 | L-alanine and D-fructose² |
| | | L-cysteine and D-fructose² |
| Bacillus megaterium | SB3112 | L-alanine, KBr, and D-glucose |
| | | L-histidine, KBr, and D-glucose |
| | | L-isoleucine, KBr, and D-glucose |
| | | L-leucine, KBr, and D-glucose |
| | | L-phenylalanine, KBr and |
| | | D-glucose |
| | | L-proline, KBr, and D-glucose |

¹Each line entry indicates an independent germinant or germinant combination which causes germination
²Sucrose could substitute for D-fructose

### Example 3. Effects of L-Valine Treatment on Germination of Bacillus pumilus Strain SB3189

The data presented in this example show that treatment of SB3189 spores with L-valine, a molecule not known to have germinant properties on SB3189 spores, could alter parameters of SB3189 germination when the L-valine was added prior to addition of germinants that caused germination.

As shown in Table 2, the following combinations of components, each component present at 3 mM, cause germination of Bacillus pumilus strain SB3189 spores: L-alanine and D-fructose, L-alanine and sucrose, L-cysteine and D-fructose, and L-cysteine and sucrose. As discussed in Example 2, each of the 20 essential L-amino acids, including L-valine, was tested for germinant activity in combination with individual sugars, and in combination with salts. L-valine did not show germinant activity in these studies.

Bacterial spores from Bacillus subtilis strain SB3189 were prepared as described in the first paragraph of Example 1. The spore preparations were washed in sterile 4° C. water by centrifuging (10,000×g, 1 minute), aspirating the supernatant from the pellet, and suspending the spore pellet in water. This was performed three consecutive times. Optical densities of washed spore preparations were measured in sterile water at 4° C. using a Synergy H4 Multi-Mode Reader (Bio-Tek). The spore preparations were set to 5 OD₆₀₀ units per ml. By phase-contrast microscopy, 99% of the particles in the samples appeared to be ungerminated spores.

The spores were then treated with 0.5, 3.0, or 10.0 mM of L-valine for 24 hours at 4° C. or 22° C. No germination of the spores occurred as a result of the L-valine treatment as determined by microscopy (germinated spores transition in appearance from phase-bright to phase-dark). Negative controls (0 mM L-valine) were not treated with L-valine. Spores to be used as positive controls were boiled for 2 hours to release all dipicolinic acid (see below).

Germination of spores in these studies was determined by measuring dipicolinic acid (DPA). DPA is about 10% of the mass of endospores, all of which is generally released when germination is triggered. Boiling spores generally releases all of the DPA. DPA release was detected using terbium chloride (TbCl₃), as described below.

To perform the germination assays, 30 μl of spores were added to wells of a 96-well flat-bottom microtiter plate. Subsequently, 35 μl of a 250 μM TbCl₃ stock was added. At time 0, 85 μl of a 10 mM L-alanine+10 mM AGFK (10 mM AGFK is 10 mM each of L-asparagine, D-glucose, D-fructose, and KCl) (10 mM AAGFK) was added. The solution was mixed by pipetting, the microtiter plate was placed into a plate reader (Synergy H4 Multi-Mode Reader, Bio-Tek), and the sample wells were measured for presence of a fluorescent Tb-DPA product over time. Tb-DPA is excited at 270 nm and emits at 545 nm. All experimental samples were tested in triplicate. Baseline controls for each sample were generated by omitting L-alanine and these were performed in duplicate. All sample wells were measured immediately after D-fructose was added (time 0) and then over time. For each time point, the data were normalized to values for the spores that had been boiled for two hours to force 100% release of DPA from the spores.

The data are shown in Tables 3 and 4. The data from Table 4 (L-valine at 22° C.) is plotted in FIG. 6.

TABLE 3
Effects of L-valine treatment at 4° C. on germination of
Bacillus pumilus strain SB3189 spores

| Concentration of L-valine | T_{lag}¹ | |
|---|---|---|
| (mM) | (minutes:seconds) | Gₘₐₓ¹ (%) |
| 0 | 7:21 | 72 |
| 0.5 | 7:21 | 74 |
| 3.0 | 7:21 | 70 |
| 10.0 | 7:21 | 76 |

¹Values are means of 3 replicates

TABLE 4
Effects of L-valine treatment at 22° C. on germination of
Bacillus pumilus strain SB3189 spores

| Concentration of L-valine | T_{lag}¹ | |
|---|---|---|
| (mM) | (minutes:seconds) | Gₘₐₓ¹ (%) |
| 0 | 7:21 | 16 |
| 0.5 | 7:21 | 6 |
| 3.0 | 7:21 | 43 |
| 10.0 | 7:21 | 77 |

¹Values are means of 3 replicates

These data show that, when L-valine was incubated with SB3189 spores at 4° C., there were small effects on germination parameters. However, when L-valine was incubated with SB3189 spores at 22° C., there were effects on germination parameters. For spores incubated with 0.5 mM L-valine at 22° C., the Gₘₐₓ decreased. At 3.0 mM L-valine, there was almost a 3-fold increase in Gₘₐₓ. At 10.0 mM L-valine, there was almost a 5-fold increase in Gₘₐₓ, as compared to the control that did not contain L-valine. T_{lag} was not changed in these studies.

### Example 4. Effects of L-Asparagine on Germination of Bacillus megaterium Strain SB3112 Spores

Table 2 indicates that spores from Bacillus megaterium SB3112 have multiple combinations of molecules that can cause germination. L-asparagine was not shown to have germinant behavior in these studies. The studies described here show that spore treatment with L-asparagine, followed by treatment with a full complement of germinants, affects parameters of germination, as compared to spores not pretreated with L-asparagine.

The studies were generally performed as described in Example 3. SB3112 spores, heat-activated or not heat-activated (not heat-activated spores are called “native”) were treated with 0.5, 3.0, or 10.0 mM L-asparagine overnight at either 4° or 22° C. Subsequently, a solution of 50 mM KBr, 20 mM D-glucose, 20 mM L-proline, 20 mM L-asparagine, and 20 mM L-valine was added (this solution includes the minimal germination requirements for SB3112 spores, as indicated in Table 2). The time at which this solution was added to the spores was considered time 0. DPA concentration was measured over time to determine germination.

The data from these studies are shown in Tables 5 and 6, below.

TABLE 5
Effects of L-asparagine treatment at 4° C. on germination of native and
heat activated Bacillus megaterium strain SB3112 spores

| Concentration | | | | |
|---|---|---|---|---|
| of L- | Native spores | | Heat activated spores | |
| asparagine | T_{lag}¹ | Gₘₐₓ¹ | T_{lag}¹ | |
| (mM) | (minutes:seconds) | (%) | (minutes:seconds) | Gₘₐₓ¹ (%) |
| 0 | 19:30 | 23 | 7:30 | 92 |
| 0.5 | 55:30 | 29 | 7:30 | 106 |
| 3.0 | 19:30 | 28 | 7:30 | 109 |
| 10.0 | 15:30 | 29 | 7:30 | 114 |

¹Values are means of 3 replicates

TABLE 6
Effects of L-asparagine treatment at 22° C. on germination of native and
heat activated Bacillus megaterium strain SB3112 spores

| Concentration | | | | |
|---|---|---|---|---|
| of L- | Native spores | | Heat activated spores | |
| asparagine | T_{lag}¹ | Gₘₐₓ¹ | T_{lag}¹ | |
| (mM) | (minutes:seconds) | (%) | (minutes:seconds) | Gₘₐₓ¹ (%) |
| 0 | 7:30 | 57 | 7:30 | 87 |
| 0.5 | 7:30 | 56 | 11:30 | 81 |
| 3.0 | 7:30 | 100 | 7:30 | 102 |
| 10.0 | 7:30 | 81 | 7:30 | 79 |

¹Values are means of 3 replicates

The data show that, at 4° C., native and heat-activated spores pretreated with all tested concentrations of L-asparagine had an increased Gₘₐₓ as compared to spores not pretreated with L-asparagine. At 22° C., pretreatment of spores with 3.0 mM of L-asparagine increased Gₘₐₓ for both native and heat-activated spores. At 22° C., pretreatment of spores with 10 mM L-asparagine also increased Gₘₐₓ for native spores.

For these spores, heat-activation generally resulted in an increased Gₘₐₓ, even without L-asparagine pretreatment. At 4° C., heat-activation also decreased T_{lag}.

While example compositions, methods, and so on have been illustrated by description, and while the descriptions are in considerable detail, it is not the intention of the applicants to restrict or in any way limit the scope of the application. It is, of course, not possible to describe every conceivable combination of components or methodologies for purposes of describing the compositions, methods, and so on described herein. Additional advantages and modifications will readily appear to those skilled in the art. Therefore, the invention is not limited to the specific details and illustrative examples shown and described. Thus, this application is intended to embrace alterations, modifications, and variations that fall within the scope of the application. Furthermore, the preceding description is not meant to limit the scope of the invention.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

1. A first composition, comprising:
a population of bacterial spores and a non-germinant contacting the bacterial spores;
the population of bacterial spores in the first composition able to germinate when contacted with sufficient germinants in water;
where, when the population of bacterial spores in the first composition is contacted with the sufficient germinants, and germination of the population of bacterial spores occurs, a parameter of the germination is different as compared to germination of the population of bacterial spores in a second composition that does not contain the non-germinant when contacted with the sufficient germinants.

2. The first composition of claim 1, where the parameter of the germination that is different indicates more efficient germination of the population of bacterial spores in the first composition.

3. The first composition of claim 1, where the non-germinant excludes molecules toxic to, or capable of killing, the population of bacterial spores, and excludes known inhibitors of germination.

4. The first composition of claim 1, where the non-germinant is a contextual non-germinant.

5. The first composition of claim 1, where the different parameter in the first composition is a decreased T_{lag}, decreased germination heterogeneity, increased Gₘₐₓ, or increased Gᵣₐₜₑ, as compared to the second composition.

6. The first composition of claim 1, where the first composition includes a partial complement of germinants.

7. The first composition of claim 1, where the first composition includes a full complement of germinants and is dry.

8. The first composition of claim 1, where the non-germinant includes an L-amino acid, salt, purine or nucleoside, vitamin, or sugar.

9. The first composition of claim 1, where the non-germinant includes a first L-amino acid that is a contextual non-germinant where a second amino acid is a germinant for the population of bacterial spores.

10. The first composition of claim 9, where the first L-amino acid includes L-valine or L-asparagine.

11. The first composition of claim 1, where the composition is dry.

12. The first composition of claim 1, where the composition is a liquid.

13. The first composition of claim 1, where the bacterial spores are from bacteria from the genera Acetonema, Actinomyces, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Coxiella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halobacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, or Seinonella.

14. The first composition of claim 1, where the bacterial spores are from the bacteria Bacillus amyloliquefaciens, Bacillus pumilis, Bacillus megaterium, or Bacillus subtilis.

15. The first composition of claim 1, where the non-germinant is present at a concentration of at least about 0.01, 0.05, 1, 2, 3, 4, 5, 6, 7, 8, 10, 15, 20, 25, 30, 35, 40, 45, 50, 60, 70, 80, 90, or 100 mM.

16. A method, comprising:
contacting a population of bacterial spores with amounts of one or more non-germinants for the bacterial spores;
the amounts of the one or more non-germinants known to contribute to more efficient germination of the population of bacterial spores, when sufficient germinants are added in water, as compared to germination of the population of bacterial spores, without the one or more non-germinants, when sufficient germinants are added in water;
the population of bacterial spores and the one or more non-germinants forming a mixture.

17. The method of claim 16, where more efficient germination means that the population of bacterial spores in the composition in contact with the one or more substances germinates with a decreased T_{lag}, decreased germination heterogeneity, increased Gₘₐₓ, or increased Gᵣₐₜₑ, as compared to the population of bacterial spores in a composition not in contact with the one or more substances.

18. The method of claim 16, including contacting the population of bacterial spores in the mixture with a partial complement of germinants.

19. The method of claim 16, including contacting the population of bacterial spores in the mixture with a full complement of germinants.

20. The method of claim 19, where the contacting of the population of bacterial spores in the mixture with a full complement of germinants occurs at least about 6 months after the contacting of the population of bacterial spores with the one or more non-germinants.
